(19) United States
(12) Patent Application Publication
(10) Pub. No.: US 2021/0070926 A1
(43) Pub. Date: Mar. 11, 2021

(54) METHOD FOR FABRICATING ABSORBENT ARTICLE

(71) Applicant: CRADERS Co.,Ltd, Seoul (KR)

(72) Inventor: Ui Cheol LEE, Seoul (KR)

(21) Appl. No.: 16/998,609

(22) Filed: Aug. 20, 2020

(30) Foreign Application Priority Data
Sep. 11, 2019 (KR) 10-2019-0112707

(51) Int. Cl. C08G 61/12; A61F 13/15; C08K 3/04; C08G 73/06; A61F 13/42

(52) U.S. Cl. CPC C08G 61/12; A61F 13/15577; C08K 3/041; C08K 3/042; C08G 73/0605; A61F 2013/424; C08G 2261/3247; C08G 2261/40; C08G 2261/50; C08K 2201/001; A61F 13/42

(57) ABSTRACT

A method for fabricating an absorbent article on which a conductive paste with good conductivity and adhesion is printed or coated and which is capable of receiving excretions discharged from a user's body is proposed. The method can include: a PEDOT dispersion polymerization step in which a 3,4-ethylenedioxythiophene (EDOT) monomer is polymerized using a dispersion polymerization method to produce poly(3,4-ethylenedioxythiophene) (PEDOT) nanoparticles; a step in which, after polymerization of the PEDOT nanoparticles is completed, residual dispersion stabilizer and oxidizer are removed and the PEDOT nanoparticles are recovered; a step in which the recovered PEDOT nanoparticles are produced into a conductive paste; and a step in which the conductive paste based on the PEDOT nanoparticles is printed or coated.

### CROSS-REFERENCE TO RELATED APPLICATION

This application claims priority to and the benefit of Korean Patent Application No. 10-2019-0112707, filed on Sep. 11, 2019, the disclosure of which is incorporated herein by reference in its entirety.

### BACKGROUND

### 1. Field of the Invention

The present invention relates to a method for fabricating an absorbent article on which a conductive paste with good conductivity and adhesion is printed or coated and which is capable of receiving excretions discharged from a user's body.

### 2. Discussion of Related Art

Diapers are absorbent articles used by the elderly, the physically disabled, or the like, and examples thereof include disposable diapers, training pants, and incontinence pants, all of which are used separately according to the lifestyle of the wearer or the level of nursing.

For example, disposable diapers are used as an aid for excretion or the like or as a sanitary product for nursing and hold and absorb excretions and prevent leakage thereof for people with difficulty in excreting by themselves, like the elderly or the physically disabled.

Urine may be an important source of information for understanding health conditions of users. Thus, there is a need for a means to sense and manage a user's urine and a way of fabricating the means.

A disposable diaper that allows for easy withdrawal of the penis for urination has been disclosed in Korean Patent Registration Publication No. 1246464, but a means to manage urine and a way of fabricating the means are not disclosed therein.

As an absorbent article, a smart diaper including a sensing means should be able to immediately receive urination information, and a conductive paste formed on the smart diaper is used therefor. Thus, the conductive paste should have excellent conductivity, and a process of forming the conductive paste should be simple.

### RELATED ART DOCUMENT

### Patent Document

(Patent Document 0001) Korean Patent Registration Publication No. 1246464

### SUMMARY OF THE INVENTION

As an absorbent article, a smart diaper including a sensing means should be able to immediately receive urination information, and a conductive paste formed on the smart diaper is used therefor. Thus, the conductive paste should have excellent conductivity, and a process of forming the conductive paste should be simple.

The present invention is directed to providing a method for fabricating an absorbent article on which a conductive paste with high heat resistance and conductivity is printed or coated and which serves as a sensor that senses a user's excretions.

The present invention provides a method for fabricating an absorbent article, the method including: a poly(3,4-ethylenedioxythiophene) (PEDOT) dispersion polymerization step in which a 3,4-ethylenedioxythiophene (EDOT) monomer is polymerized using a dispersion polymerization method to produce PEDOT nanoparticles; a step in which, after polymerization of the PEDOT nanoparticles is completed, residual dispersion stabilizer and oxidizer are removed and the PEDOT nanoparticles are recovered; a step in which the recovered PEDOT nanoparticles are produced into a conductive paste; and a step in which the conductive paste based on the PEDOT nanoparticles is printed or coated.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a flowchart showing a method for fabricating an absorbent article of the present invention;

FIG. 2 is a schematic diagram of an absorbent article fabrication apparatus of the present invention that is viewed from a side;

FIG. 3 is a schematic diagram of the absorbent article fabrication apparatus of the present invention that is viewed from the top;

FIG. 4 is a schematic diagram of an absorbent article fabricated using the method for fabricating the absorbent article of the present invention; and

FIG. 5 is a schematic diagram illustrating a cross-section of a sheet portion.

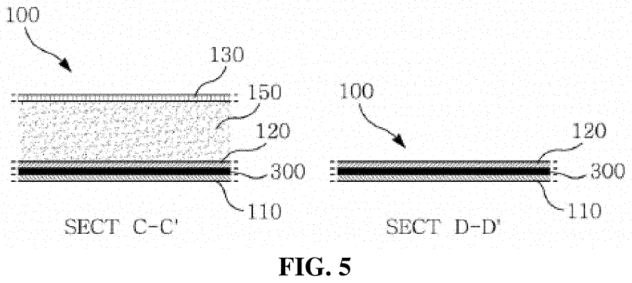

### DETAILED DESCRIPTION OF EXEMPLARY EMBODIMENTS

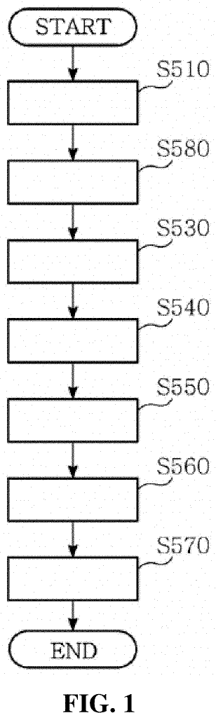

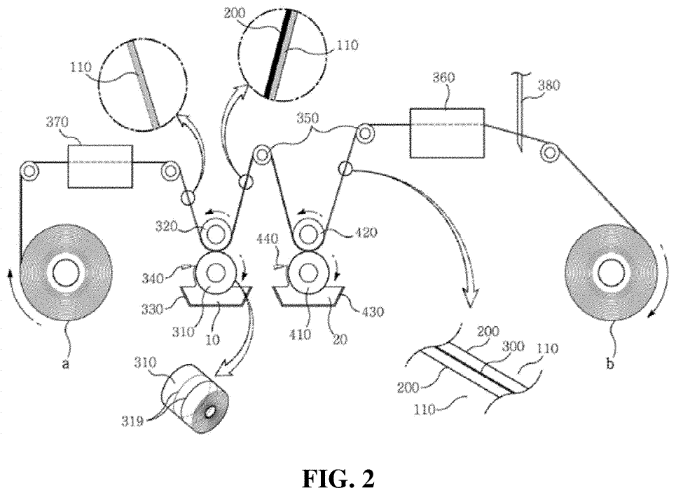

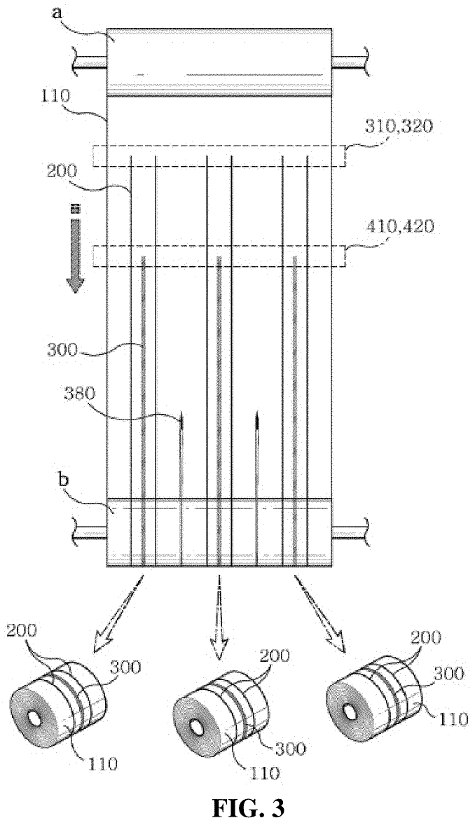

FIG. 1 is a flowchart showing a method for fabricating an absorbent article of the present invention. FIG. 2 is a schematic diagram of an absorbent article fabrication apparatus of the present invention that is viewed from a side. FIG. 3 is a schematic diagram of the absorbent article fabrication apparatus of the present invention that is viewed from the top.

The method for fabricating the absorbent article that is shown in FIG. 1 may include an unwinding step (S510) in which a first sheet 110 is provided, a preprocessing step (S580) in which the first sheet 110 is preprocessed, a sensing pattern forming step (S530) in which a sensing pattern 200 is formed on the first sheet 110, a marker line forming step (S540) in which a marker line 300 is formed on the first sheet 110, a drying step (S550) in which the sensing pattern 200 or the marker line 300 is dried, a slitting step (S560) in which the first sheet 110 having a large width is slit into a plurality of first sheets 110 each having a small width, and a winding step (S570) in which the first sheet 110, on which the sensing pattern 200 is formed, is wound to form a sensing pattern roll b.

The unwinding step (S510) corresponds to a step in which the first sheet 110 is prepared and provided.

The first sheet 110 may be formed in the shape of a plate and may include materials such as vinyl or a film that at least partially limits passage of excretions. For example, the first sheet 110 may include polyethylene.

In the absorbent article, the first sheet 110 may be disposed in an excretion area which receives a user's excretions.

The user's excretions may include feces, urine, sweat, menstrual fluid, tears, saliva, and the like.

In order to receive each type of excretion, the absorbent article may include a sheet portion 100 such as a diaper or pad that receives feces and urine, a patch attached to a specific site of a user's body to receive sweat, a sanitary pad that receives menstrual fluid, an eye patch that receives tears, and a mask that receives saliva or the like.

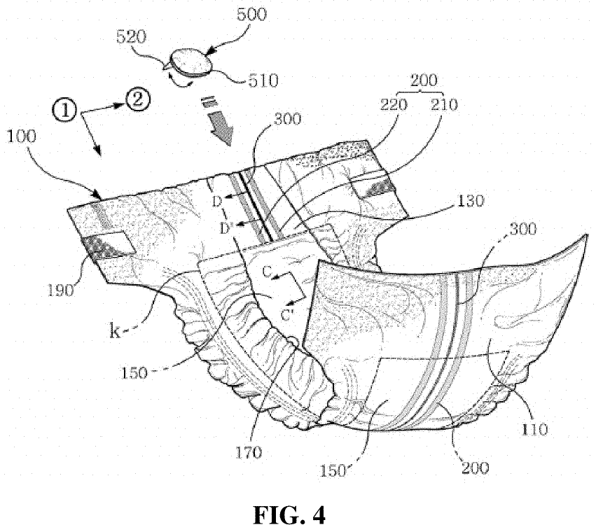

The sheet portion 100 illustrated in FIG. 4 may include a diaper fastened between a user's legs to receive feces and urine. Each configuration illustrated in FIG. 4 may be applied as it is to other types of sheet portions 100 such as a sanitary pad.

The first sheet 110 may be formed by molding polyethylene powder.

In order to improve convenience in handling, a source roll a, around which the first sheet 110 not having the sensing pattern 200 formed thereon is wound, may be prepared in the unwinding step (S510).

The unwinding step (S510) may include a step in which the plate-shaped first sheet 110 which at least partially limits passage of excretions is unwound from the source roll a around which the first sheet 110 is wound.

In the method for fabricating the absorbent article of the present invention, the fabricated first sheet 110 may be for forming the sheet portion 100 on which a terminal portion 500 may be mounted, wherein the terminal portion 500 includes terminals 531, 532, 533, and 534 configured to apply a sensing signal.

Therefore, the absorbent article of the present invention may be a so-called smart diaper. The sensing pattern 200 which physically comes in contact with the terminals of the terminal portion 500 and communicates with the sensing signal may be formed on the sheet portion 100.

Here, the sensing pattern 200 which may communicate with the sensing signal may be formed by a conductive paste 10. In order to sense excretions using the sensing signal, a plurality of terminals spaced apart from each other may be formed in the terminal portion 500. Here, a positive (+) sensing pattern 210 which comes in contact with some of the terminals and a negative (−) sensing pattern 220 which comes in contact with the other terminals may be formed on the sheet portion 100.

Since the positive sensing pattern 210 and the negative sensing pattern 220 are spaced apart from each other, a sensing signal applied to the positive sensing pattern 210 may not be transmitted to the negative sensing pattern 220 in an initial state in which there is no excretion. When excretion is distributed across the positive sensing pattern 210 and the negative sensing pattern 220, the sensing signal of the positive sensing pattern 210 may be transmitted to the negative sensing pattern 220 over the excretion. The terminal portion 500 may analyze the sensing signal transmitted to the negative sensing pattern 220 and sense the excretion or determine the amount of excretion.

For normal operation of the terminal portion 500, a thickness, a width, and the like of each sensing pattern 200 should satisfy predetermined values. However, it may be difficult to print the conductive paste 10 corresponding to the thickness and width satisfying the predetermined values on the first sheet 110 having a smooth surface.

In order to form the sensing pattern 200 having a thickness and width that satisfy the predetermined values, the preprocessing step (S580) may be performed before the sensing pattern forming step (S530) in which the sensing pattern 200 is formed.

In the preprocessing step (S580), the first sheet 110 unwound from the source roll a may be corona-treated.

For example, in the preprocessing step (S580), the plate-shaped first sheet 110 which limits passage of excretions may be corona-treated, and an angle of contact between one surface of the first sheet 110 and water may be decreased.

Corona treatment corresponds to discharge treatment and is a method in which a corona salt is generated using a discharge treatment portion 370 and the first sheet 110 is passed therebetween to modify a surface of the first sheet 110. The surface of the first sheet 110 is oxidized and the angle of contact with water, which indicates the degree of hydrophilicity, is decreased by the corona treatment. As a result, since the printability of the first sheet 110 made of polyethylene is greatly improved, the sensing pattern 200 may be precisely formed to have a thickness and width that satisfy predetermined values by using the conductive paste 10.

The discharge treatment portion 370 configured to corona-treat the first sheet 110 unwound from the source roll a may be disposed between the source roll a and sensing pattern portions 310 and 320 forming the sensing pattern 200.

The sensing pattern portions may print or coat the conductive paste on the first sheet 110, the hydrophilicity of which is improved through the preprocessing step (S580), to form the sensing pattern 200.

In the sensing pattern forming step (S530), the sensing pattern 200 that may communicate with an excretion sensing signal may be formed on the first sheet 110 unwound from the source roll a.

For example, in the sensing pattern forming step (S530), by printing or coating the conductive paste 10, which contains water, on one surface of the first sheet 110, the hydrophilicity of which is improved through the corona treatment, the sensing pattern 200 that may communicate with an excretion sensing signal may be formed.

In the sensing pattern forming step (S530), a plurality of sensing patterns 200 passing through an excretion area k of the first sheet 110 that receives excretions may be printed or coated at predetermined intervals on the first sheet 110 so that an equivalent resistance of the sensing pattern 200 changes due to the excretions. Here, each sensing pattern 200 may be printed or coated continuously on the first sheet 110 in a direction in which the first sheet 110 is unwound from the source roll a.

The sensing pattern portions forming the sensing patterns 200 on the first sheet 110 may be disposed between the source roll a and the sensing pattern roll b.

As the first sheet 110 is continuously unwound from the source roll a, the first sheet 110 may continuously pass between the sensing pattern portions.

On the first sheet 110 continuously passing between the sensing pattern portions, the sensing patterns 200 may be continuously formed by the sensing pattern portions.

The first sheet 110 that has continuously passed between the sensing pattern portions may be continuously wound around the sensing pattern roll b.

The sensing pattern roll b may have a cylindrical roll core around which the first sheet having the sensing pattern formed thereon is wound. According to circumstances, the sensing pattern roll b may be formed without the roll core.

The first sheet 110 wound around the sensing pattern roll b may be cut to fit the length of the absorbent article by a cutting portion in a post-process for fabricating the absorbent article. In reality, it is difficult to wind the first sheet 110 of infinite length around the sensing pattern roll b. The first sheet 110 may be cut into pieces of predetermined lengths and, even in this case, the first sheet may be cut by the cutting portion. In a case in which the first sheet wound around the source roll a has a limited length, the sensing pattern roll b around which the first sheet having the limited length is wound may be formed without using a separate cutting portion.

Here, in order to allow each of the plurality of sensing patterns to come in contact with one of the plurality of terminals formed in the terminal portion, which is configured to generate sensing signals, regardless of sites at which the first sheet 110 is cut, the plurality of sensing patterns 200 may be formed to be parallel at equal intervals in the sensing pattern forming step (S530).

In order to allow an end of the cut first sheet 110 and an end of the sensing pattern 200 formed on the cut first sheet 110 to match regardless of sites at which the first sheet 110 is cut, the sensing patterns 200 may be continuously formed in a longitudinal direction of the first sheet 110 in the sensing pattern forming step (S530).

In a case in which the sensing patterns 200 include a first sensing pattern and a second sensing pattern spaced apart from each other, both an end of the first sensing pattern and an end of the second sensing pattern may be disposed at the end of the cut first sheet 110. For example, the first sensing pattern may include the positive sensing pattern 210 of FIG. 4, and the second sensing pattern may include the negative sensing pattern 220 of FIG. 4.

Here, in the sensing pattern forming step (S530), the plurality of sensing patterns 200 spaced apart from each other may be formed corresponding to intervals at which the plurality of terminals are formed in the terminal portion 500 configured to generate sensing signals for sensing excretions.

For example, the sensing patterns 200 may be formed on the first sheet 110 by gravure printing.

The sensing pattern forming step (S530) may be performed by the sensing pattern portions disposed between the source roll a and the sensing pattern roll b.

The sensing pattern portions may include a first cylindrical plate gravure press 310 provided on a movement path of the first sheet 110 moving from the source roll a toward the sensing pattern roll b and configured to rotate in accordance with movement of the first sheet 110.

In the sensing pattern forming step (S530), using a first flat plate gravure press 320 and the first cylindrical plate gravure press 310 provided on the movement path of the first sheet 110, the sensing patterns 200 may be printed or coated continuously on the first sheet 110 input to the first flat plate gravure press 320 and the first cylindrical plate gravure press 310.

A first ink container 330 accommodating the conductive paste 10 in a liquid or powder form, the first cylindrical plate gravure press 310 having an outer circumferential surface on which the sensing pattern 200 is formed in an engraved manner and which rotates as the first cylindrical plate gravure press 310 passes through the first ink container 330, and the first flat plate gravure press 320 disposed to face the first cylindrical plate gravure press 310 may be provided. A plurality of trenches 319 corresponding to a plurality of engraved ribs, which are spaced apart at intervals equal to intervals between the plurality of terminals, may be formed on the outer circumferential surface of the first cylindrical plate gravure press 310.

Here, the plurality of trenches 319 may extend parallel to each other.

Regardless of whether the trench 319 is tilted in the longitudinal direction of the first sheet 110, the trench 319 may be formed in the shape of a closed curve in which the start and end are connected on the circumferential surface of the first cylindrical plate gravure press 310. The plurality of trenches 319 may be formed to be parallel to each other at equal intervals in a width direction of the first sheet 110.

Each trench 319 may be formed continuously without interruption along the outer circumference of the first cylindrical plate gravure press 310. The conductive paste in the trench 319 may be printed or coated on one surface of the first sheet 110.

Due to the trenches 319, the plurality of sensing patterns 200 parallel to each other may be formed continuously without interruption on the first sheet 110 that has passed through the first cylindrical plate gravure press 310 while being pressed against the rotating first cylindrical plate gravure press 310.

For example, the conductive paste 10 is held in the plurality of trenches 319 that pass through the first ink container 330, and the corresponding conductive paste 10 may be printed on the first sheet 110 facing the plurality of trenches 319. The plurality of sensing patterns 200 printed on the first sheet 110 by the engraved ribs may be spaced apart at intervals equal to the intervals between the plurality of terminals.

A first doctor blade 340 configured to adjust a thickness of the conductive paste 10 on the outer circumferential surface and a platen roller 350 configured to maintain the first sheet 110 taut may be further provided at positions facing the first cylindrical plate gravure press 310.

The first sheet 110 unwound from the source roll a may be input between the first cylindrical plate gravure press 310 and the first flat plate gravure press 320 and may be printed or coated with the conductive paste 10 between the presses. Here, the conductive paste 10 printed or coated on one surface of the first sheet 110 may form the sensing pattern 200, which may communicate with the sensing signal, and be naturally dried or forcibly dried by a drying portion 360. The first sheet 110 on which the conductive paste 10 is dried is the first sheet 110 having the sensing pattern 200 formed thereon, and the first sheet 110 having the sensing pattern 200 formed thereon may be rewound in a roll.

When the source roll a around which the first sheet 110 is wound is disposed at a first position and the sensing pattern roll b is disposed at a second position, the first sheet 110 unwound from the source roll a may continue to the second position and be wound around the sensing pattern roll b at the second position.

Here, the preprocessing step (S580), the sensing pattern forming step (S530), the marker line forming step (S540), the drying step (S550), and the slitting step (S560) may be performed between the first position and the second position and performed on the first sheet 110 that is continuous from the first position to the second position.

According to the present embodiment, the first sheet 110 may be input in a state of being wound around the source roll, and the first sheet 110 on which various steps are performed may be output to be wound around the sensing pattern roll.

In the marker line forming step (S540), a marker line that changes color when the first sheet 110 is stained with excretion may be formed. The marker line forming step (S540) may be performed by marking portions 410 and 420 which are disposed between the sensing pattern portions configured to perform the sensing pattern forming step (S530) and the source roll a or disposed between the sensing pattern portions and the sensing pattern roll b.

A second ink container 430 accommodating hydrochromic ink 20 in a liquid or powder form, a second cylindrical plate gravure press 410 having an outer circumferential surface on which the sensing pattern 200 is formed in an engraved manner and which rotates as the second cylindrical plate gravure press 410 passes through the second ink container 430, and a second flat plate gravure press 420 disposed to face the second cylindrical plate gravure press 410 may be provided. A second doctor blade 440 configured to adjust a thickness of the hydrochromic ink 20 on the outer circumferential surface and the platen roller 350 configured to maintain the first sheet 110 taut may be further provided at positions facing the second cylindrical plate gravure press 410.

The marking portions such as the second flat plate gravure press 420 that performs the marker line forming step (S540) may be disposed in front of or behind the sensing pattern portions such as the first flat plate gravure press 320 that performs the sensing pattern forming step (S530). Therefore, the marker line forming step (S540) may be performed before or after the sensing pattern forming step (S530). Referring to FIG. 1, the marker line forming step (S540) is performed after the sensing pattern forming step (S530).

In the marker line forming step (S540), a guide portion 521 of the terminal portion 500 may be formed to guide alignment between the terminals of the terminal portion 500 and the sensing patterns 200 on the first sheet 110, and a plurality of marker lines 300 may be formed corresponding to the intervals between the terminals so as to be spaced apart from the sensing patterns 200.

In the drying step (S550), the conductive paste 10, which is formed on one surface of the first sheet 110 after the sensing pattern forming step, may be forcibly dried using the drying portion 360.

The drying portion 360 may use a heater, hot air, or the like to dry the conductive paste 10 or hydrochromic ink 20 on the sensing patterns 200. The drying portion 360 may be disposed between the sensing pattern portions and the sensing pattern roll b. The drying portion 360 may dry the sensing patterns 200 formed on the first sheet 110 before the first sheet 110 is wound around the sensing pattern roll b.

The drying step (S550) may be performed on the first sheet 110 after both the sensing pattern forming step (S530) and the marker line forming step (S540) are completed. In addition, the drying step (S550) may be performed in between the sensing pattern forming step (S530) and the marker line forming step (S540).

The preprocessing step (S580), the sensing pattern forming step (S530), and the marker line forming step (S540) may be performed on the first sheet 110 having a width larger than or equal to an integer multiple of the width of the absorbent article that receives excretions.

In the sensing pattern forming step (S530) or the marker line forming step (S540), the sensing patterns 200 or the marker lines 300 may be formed at every area or position corresponding to each absorbent article in the width direction of the first sheet 110.

In the slitting step (S560) after the sensing pattern forming step (S530) and the marker line forming step (S540), the first sheet 110 having a width larger than or equal to an integer multiple of the width of the absorbent article may be slit using a slitting portion to form a plurality of first sheets 110 each having a width necessary for fabricating a single absorbent article.

When the first sheet 110, on which the sensing pattern forming step (S530) or the marker line forming step (S540) is performed, is wound to form the sensing pattern roll b, the slitting step (S560) may be performed using a slitting portion 380, such as a cutter, provided between the sensing pattern portions configured to perform the sensing pattern forming step (S530) and the sensing pattern roll b or may be performed using the slitting portion 380 provided between the marking portions configured to perform the marker line forming step (S540) and the sensing pattern roll b.

According to circumstances, the slitting step (S560) may also be performed by cutting the sensing pattern roll b.

The slitting portion 380 may cut a first sheet 110 having a first width, which moves from the sensing pattern portions toward the sensing pattern roll between the sensing pattern portions and the sensing pattern roll, to form a plurality of first sheets 110 each having a second width smaller than the first width. Alternatively, the slitting portion 380 may cut a first sheet 110 having a first width, which moves from the marking portions toward the sensing pattern roll between the marking portions and the sensing pattern roll, to form a plurality of first sheets 110 each having a second width smaller than the first width.

The first sheet 110 cut into a plurality of pieces in the longitudinal direction by the slitting portion 380 may be wound around the sensing pattern roll b in the winding step (S570).

The first sheet 110 having the second width due to the slitting portion 380 may be transferred to a subsequent step for fabricating the absorbent article while being wound around the sensing pattern roll b having the second width.

When the first sheet 110, on which the sensing pattern forming step (S530) is performed, is wound to form the sensing pattern roll, a sub-roll around which a second sheet 120, which is made of a material that allows passage of excretions through the subsequent step for fabricating the absorbent article, is wound may be provided.

The sensing pattern roll and the sub-roll may be installed such that the second sheet 120 unwound from the sub-roll faces one surface of the first sheet 110 on which the sensing patterns 200 are printed or coated that is unwound from the sensing pattern roll.

The first sheet 110 and the second sheet 120 may enter a joining roller together so that the second sheet 120 faces one surface of the first sheet 110 on which the sensing patterns 200 are printed or coated.

Using the joining roller, the second sheet 120 may be joined with one surface of the first sheet 110 on which the sensing patterns 200 are printed or coated.

The terminals of the terminal portion may be pressed against the second sheet 120, pass through the second sheet, and come in contact with the sensing patterns on the first sheet.

In a case in which the first sheet includes a material that allows passage of a portion of excretions, the terminals of the terminal portion may be directly pressed against the first sheet 110 and come in contact with the sensing patterns on the first sheet.

The first sheet 110 and the second sheet 120 output from the joining roller and joined with each other may be cut by a cutting portion to fit the length of the absorbent article that receives excretions.

The first sheet 110 and the second sheet 120 cut to fit the length of the absorbent article may be sewn to correspond to the absorbent article.

An absorbent material 150 configured to absorb excretions may be laminated on the center of the second sheet 120 which is sewn to correspond to the absorbent article and joined with the first sheet 110.

When the absorbent material 150 laminated on the center of the second sheet 120 is covered with a third sheet 130 made of a material that allows passage of excretions and an edge of the third sheet 130 is joined with an edge of second sheet 120, the sheet portion 100 that corresponds to the absorbent article having the sensing patterns 200 formed thereon may be completed.

FIG. 4 is a schematic diagram of an absorbent article fabricated using the method for fabricating the absorbent article of the present invention.

In FIG. 4, a diaper fastened between a user's legs to receive feces and urine is illustrated as an example of the sheet portion 100 that corresponds to the absorbent article.

A pair of tape fasteners 190 may be installed at one-side edge of the diaper. A tape of the tape fasteners 190 may be attached to the other-side edge of the diaper.

A pair of leakage-preventing cuffs 170 may be provided at both sides at the center of the diaper so as to stand upright.

The absorbent material 150 may include materials such as cotton, a super absorption polymer (SAP), and tissue.

Here, the conductive paste 10 printed or coated on one surface of the first sheet 110 of the absorbent article may be fabricated as follows using the method for fabricating the absorbent article of the present invention.

The conductive paste 10 may include at least one of materials including metal oxides, such as carbon, through which electricity is communicated, and conductive polymers (conductive materials such as a carbon-based material, a polymer-based material, metal oxide, and PEDOT), a solution for diluting the conductive materials such as the carbon-based material, polymer-based material, metal oxide, and PEDOT, and water and acetyl corresponding to a solvent of the conductive polymer.

The PEDOT is poly(3,4-ethylenedioxythiophene).

Polymers may be largely divided into performance polymers and functional polymers. The performance polymers are derived from the properties of polymers and characterized by mechanical or thermal properties. The functional polymers refer to polymers that express unique properties according to a functional group present in the polymer or express the polymers' function as a functional material is blended into the polymer.

Conductive polymers are mainly used for conductive molding materials and conductive films and are used in various fields such as transportation of integrated circuits (IC) and transparent electrodes. The various fields may also include a technology for blending a conductivity-imparting agent into a polymer.

The conductive polymers may include polyacetylene, polypyrrole, polythiophene, polyaniline, and the like.

A specific method for fabricating the conductive paste 10 is as follows.

There may be provided a method for fabricating an absorbent article, the method including: (A) a PEDOT dispersion polymerization step in which an EDOT monomer is polymerized using a dispersion polymerization method to produce PEDOT nanoparticles; (B) a step in which, after polymerization of the PEDOT nanoparticles is completed, residual dispersion stabilizer and oxidizer are removed and the PEDOT nanoparticles are recovered; (C) a step in which the recovered PEDOT nanoparticles are produced into a conductive paste; and (D) a step in which the conductive paste based on the PEDOT nanoparticles is printed or coated.

The EDOT is 3,4-ethylenedioxythiophene, and the PEDOT nanoparticles may be made of PEDOT or PEDOT:polystyrene sulfonate (PSS).

In the dispersion polymerization method, a dispersion stabilizer is added to distilled water, the dispersion stabilizer is dispersed through heating and stirring, an oxidizer is added to an aqueous solution in which the dispersion stabilizer is dispersed and the aqueous solution is stirred to dissolve and disperse the dispersion stabilizer and the oxidizer, and an EDOT monomer is dropped in the aqueous solution, in which the dispersion stabilizer and the oxidizer are dispersed, to perform polymerization.

By adding a solvent to a solution in which polymerization is completed according to the dispersion polymerization method, it is possible to perform step (B) in which the residual dispersion stabilizer and oxidizer are dissolved to recover the PEDOT nanoparticles.

Preferably, the amount of the EDOT monomer used in step (A) may be 0.2 to 0.6 mol with respect to water.

The dispersion stabilizer used in step (B) is not particularly limited and any of polyvinyl alcohol, polyvinyl pyrrolidone, and polyvinyl acetate may be used as the dispersion stabilizer, but polyvinyl alcohol may be preferable in terms of production speed and commercialization. A molecular weight of the dispersion stabilizer or a concentration of the dispersion stabilizer in water is a variable for a diameter of the produced nanoparticles.

Also, examples of the oxidizer used in step (B) include iron(III) chloride (FeCl₃) and copper(II) chloride (CuCl₂). One of FeCl₃ and CuCl₂ may be selected as the oxidizer according to conditions. When polymerizing the EDOT monomer, it is preferable to use FeCl₃ as the oxidizer. This is because FeCl₃ not only provides an active site as an oxidizer but also acts as a dopant. FeCl₃ may be added 1 to 13 times more at a molar ratio as compared to a pyrrole monomer, and the electrical conductivity and yield may be adjusted accordingly.

Also, when removing the residual dispersion stabilizer and oxidizer in step (B), by adding the solvent to the solution in which polymerization is completed, the residual dispersion stabilizer and oxidizer may be dissolved to recover the PEDOT nanoparticles.

Therefore, when the residual dispersion stabilizer and oxidizer are removed, nanometer-diameter particles are obtained. The type and amount of solvent used when removing the residual dispersion stabilizer and oxidizer are not particularly limited.

However, it is preferable to use a solvent that melts both the dispersion stabilizer and oxidizer and is not toxic to humans or harmful to the environment. When polyvinyl alcohol is used as the dispersion stabilizer, it may be preferable to use distilled water as the solvent.

When polyvinyl alcohol is used as the dispersion stabilizer, it is preferable to use distilled water as the solvent used when removing the residual dispersion stabilizer and oxidizer. However, the type or amount of solvent is not particularly limited.

By removing the solvent added to the solution in which polymerization is completed according to the dispersion polymerization method, PEDOT nanoparticles may be recovered. in a solid powder form.

Here, a temperature at which the solvent is removed and a method for removing the solvent are not particularly limited, but when distilled water is used as the solvent, drying is performed at a temperature between room temperature and 80° C. Alternatively, a vacuum oven may be used to remove the solvent.

The method for fabricating the absorbent article may further include mixing the recovered PEDOT nanoparticles with a binder resin, a conductive additive, and a solvent to fabricate the conductive paste.

The solvent may be one selected from the group consisting of distilled water, ethanol, and acetone, or a mixture of two or more thereof.

The binder resin may be dissolved in a solvent and used. Here, the type or molecular weight of the binder resin is not particularly limited, and the type of solvent is also not particularly limited.

However, when the molecular weight of the binder resin is excessively high or low, adhesion to the first sheet of the absorbent article may be degraded. The concentration of the dissolved binder resin is also not particularly limited but may act as a variable for the degree of mixing or viscosity in fabricating the conductive paste.

The solvent for the binder resin may be one selected from the group consisting of dimethyl formamide, 1-methyl-2-pyrrolidone, ethylene glycol butyl ether, terpineol, toluene, and propylene glycol diacetate, or a mixture of two or more thereof.

In the paste, the amount of conductive polymer may be 0.1 to 20 wt % with respect to the weight of a binder resin solution.

The concentration of the binder resin solution may be 2 to 20 wt % with respect to the weight of the solvent.

When the ratio of the binder resin with respect to the PEDOT nanoparticles is excessively high, the adhesion to the first sheet of the absorbent article may be improved but the conductivity of the paste may be decreased. Also, when the ratio of the binder resin with respect to the PEDOT nanoparticles is excessively low, the conductivity of the paste is improved, but the adhesion to the first sheet of the absorbent article is not maintained to be high. Also, the type of solvent for the binder resin may be different according to the type of the binder resin.

The conductive additive may be one selected from the group consisting of graphite, a multi-wall carbon nanotube, and expanded graphite, or a mixture of two or more thereof.

Examples of the conductive additive include graphite and a carbon-based material such as a carbon nanotube. One of graphite and carbon nanotube may be selected as the conductive additive according to conditions. The content of the conductive additive is not particularly limited. However, when the content of the conductive additive is excessively high, the viscosity increases and mixing is difficult, and when the molecular weight of the conductive additive is excessively low, the viscosity or conductivity of the paste may be decreased.

A thickness of the PEDOT nanoparticles may be in a range of 10 to 200 nm.

Meanwhile, the conductive paste according to the present invention may include polypyrrole nanoparticles produced by polymerizing the pyrrole monomer using the dispersion polymerization method.

The above-mentioned PEDOT may be obtained by polymerizing 3,4-ethylenedioxythiophene (EDOT) by chemical polymerization or the like. Since the PEDOT itself is insoluble, a PEDOT-based conductive material may be dissolved in an organic solvent or dispersed in an aqueous solvent by using a formula according to a substituent or using an aqueous polymer dopant. PEDOT:PSS is a water-dispersible polythiophene derivative formed of PEDOT and polystyrene sulfonate (PSS).

PEDOT:PSS has excellent properties such as being dispersible in water, high transparency and conductivity, and excellent heat resistance and safety. PSS and PEDOT compensate charges for each other, and a PEDOT oligomer is strongly bound to a PSS polymer chain. The compounds may be mixed with each other and dispersed in an aqueous solution as gel particles.

Thin film properties of PEDOT:PSS may vary significantly according to whether an additive is present. When there is no additive, the conductivity of PEDOT:PSS is about 10 S/cm, which is very low. However, when, for example, dimethyl sulfoxide (DMSO), which is a high-boiling-point solvent, is added at 5% when forming a film, the conductivity may be improved by as much as about 100 times.

A polyolefin resin, especially low-density polyethylene (LDPE), has low crystallinity and excellent processability and flexibility and thus is commonly utilized as materials for diapers, but since the surface energy is about 30 mJ/m2, which is relatively low, it may be difficult for the conductive paste, which is an electrode material for smart diapers, to maintain adhesion.

In order to overcome such a problem, in the present invention, the PEDOT nanoparticles are more rapidly recovered after being produced, and the conductive paste based on the PEDOT nanoparticles is fabricated and applied to absorbent articles such as smart diapers so that the absorbent articles may have good conductivity and adhesion.

In order to improve sensitivity of sensing urine by voltage or current, the electrical resistance of the conductive paste is preferably in a range of 0.001 to 10000 MΩ. It is preferable to decrease the resistance as much as possible so that the voltage or current applied to a pattern for measurement is minimized and a change in an output signal is maximized. Since polymers are basically non-conductors, measurement sensitivity may be increased by adding a conductive additive such as carbon and minimizing the overall electrical resistance of the conductive paste composition to 0.001 to 10000 MΩ.

According to the method for fabricating an absorbent article of the present invention, a sensing pattern through which a sensing signal of a terminal portion may flow can be formed on the absorbent article that receives excretions.

In order to prevent leakage of the excretions, a first sheet made of a material that at least partially limits passage of the excretions may be provided in the absorbent article.

The first sheet constituting the absorbent article has a smooth surface, and thus it may be difficult for a conductive paste forming the sensing pattern to be adsorbed onto the first sheet. When the conductive paste is not adsorbed normally, it is difficult to expect excretions to be sensed by the sensing signal flowing through the sensing pattern.

Therefore, in order to accurately form predetermined intervals, predetermined widths, and a sensing pattern that satisfies the predetermined widths, there is a need to improve the degree of adhesion (or degree of adsorption) of the conductive paste to the first sheet.

According to the method for fabricating the absorbent article of the present invention, the degree of adhesion and conductivity of the conductive paste printed on one surface of the first sheet can be improved.

PEDOT nanoparticles are synthesized and then rapidly recovered, and a conductive paste based on the PEDOT nanoparticles is produced such that the conductive paste maintains good conductivity and adhesion. Thus, according to the present invention, the conductive paste in a good state can be printed or coated on the absorbent article.

Since the PEDOT nanoparticles are synthesized and then rapidly recovered (high recovery rate) and used for the absorbent article, the conductive paste can have high adhesion, and thus reliability of the absorbent article can be improved.

Removing a dispersion stabilizer used when producing the PEDOT nanoparticles is facilitated, the PEDOT nanoparticles can be produced uniformly and recovered rapidly, and the viscosity of the conductive paste can be controlled by adding or removing additives.

In the method for fabricating the absorbent article of the present invention, a source roll at a first position, around which the first sheet is wound, can be unwound and rewound around a sensing pattern roll at a second position, and the first sheet that is continuous between the source roll and the sensing pattern roll can be treated with the sensing pattern. Therefore, according to the present invention, since a roll-to-roll method is used in which the source roll is input and the sensing pattern roll is output, it is possible to provide convenience in that input and output of the first sheet are possible while the first sheet is in a roll.

In the method for fabricating the absorbent article of the present invention, a plurality of sensing patterns can be formed corresponding to intervals at which terminals of the terminal portion configured to sense excretions are disposed, and a marker line can be formed corresponding to a guide portion of the terminal portion. As a result, the sensing pattern of the absorbent article using the first sheet that is fabricated according to the present invention can be easily aligned with the terminals of the terminal portion by the marker line.

In the method for fabricating the absorbent article of the present invention, by forming a plurality of sensing patterns on a first sheet, a width of which is larger than that of the absorbent article, and slitting the first sheet, it is possible to simultaneously produce a plurality of first sheets for absorbent articles.

A first sheet with a sensing pattern printed or coated thereon may be wound a plurality of times around a pattern roll of the present invention. Therefore, when absorbent articles such as diapers are produced using the first sheet wound around the pattern roll, it is possible to fabricate an absorbent article having a sensing function that is capable of sensing excretions.

1. A method for fabricating an absorbent article, the method comprising the steps of:
polymerizing a 3,4-ethylenedioxythiophene (EDOT) monomer using a dispersion polymerization method to produce a poly EDOT (PEDOT) nanoparticles which are conductivity polymers;
removing residual dispersion stabilizer and oxidizer and recovering the PEDOT nanoparticles after the PEDOT nanoparticles are produced;
producing the recovered PEDOT nanoparticles into a conductive paste; and
printing or coating the conductive paste.

2. The method of claim 1, wherein the PEDOT nanoparticles are made of PEDOT:polystyrene sulfonate (PEDOT:PSS).

3. The method of claim 1, further comprising mixing the recovered PEDOT nanoparticles with a binder resin, a conductive additive, and a solvent to fabricate the conductive paste.

4. The method of claim 3, wherein the solvent is one selected from the group consisting of distilled water, ethanol, and acetone, or a mixture of two or more thereof.

5. The method of claim 3, wherein the conductive paste includes an amount of conductive polymer which is 0.1 to 20 wt % with respect to a weight of a binder resin solution.

6. The method of claim 3, wherein a concentration of a binder resin solution is 2 to 20 wt % with respect to a weight of the solvent.

7. The method of claim 3, wherein the conductive additive is one selected from the group consisting of graphite, a multi-wall carbon nanotube, and expanded graphite, or a mixture of two or more thereof.

8. The method of claim 1, wherein the conductive paste includes polypyrrole nanoparticles which are conductive polymers produced by polymerizing a pyrrole monomer using the dispersion polymerization method.

9. The method of claim 1, wherein an electrical resistance of the conductive paste is in a range of 0.001 to 10,000 MΩ.
